(19) United States
(12) Patent Application Publication
(10) Pub. No.: US 2024/0122704 A1
(43) Pub. Date: Apr. 18, 2024

(54) RECOVERABLE AND SELF-EXPANDING SHORT-FRAME VALVE SUITABLE FOR AORTIC VALVE REGURGITATION

(71) Applicant: Fuwai Hospital, Chinese Academy of Medical Sciences, Beijing (CN)

(72) Inventors: Yang Chen, Beijing (CN); Yongjian Wu, Beijing (CN); Qingrong Liu, Beijing (CN); Jie Zhao, Beijing (CN); Hongliang Zhang, Beijing (CN)

(21) Appl. No.: 18/471,549

(22) Filed: Sep. 21, 2023

(30) Foreign Application Priority Data
Oct. 14, 2022 (CN) 202211260451.X

(51) Int. Cl. A61F 2/24

(52) U.S. Cl. CPC A61F 2/2418; A61F 2220/0075; A61F 2250/0006

(57) ABSTRACT

Disclosed is a recoverable and self-expanding short-frame valve suitable for aortic valve regurgitation, including a tubular frame and valve leaflets. The tubular frame can contract to a delivery configuration or expand to a use configuration in a radial direction; the tubular frame includes a first portion and a second portion integrally formed and coaxially arranged; a waist portion is formed at the junction of the first portion and the second portion when the tubular frame is in the use configuration; and the second portion is divergently arranged in a direction away from the waist portion. According to the present disclosure, the complete recovery of the valve can be realized without affecting the later coronary intervention.

### CROSS-REFERENCE TO RELATED APPLICATIONS

This application claims priority of Chinese Patent Application No. 202211260451.X, filed on Oct. 14, 2022, the entire contents of which are incorporated herein by reference.

### TECHNICAL FIELD

The present disclosure relates to the technical field of surgical instruments for aortic valve regurgitation, in particular to a recoverable and self-expanding short-frame valve suitable for aortic valve regurgitation.

### BACKGROUND

At present, transcatheter aortic valve replacement (TAVR) has been widely applied in patients with severe aortic valve stenosis, and has become as important as surgical thoracotomy and valve replacement. Moreover, TAVR technology is more minimally invasive and contributes to the faster recovery of patients. That being said, at present, there are still many problems to be solved when it comes to direct application of the TAVR technology to aortic valve regurgitation.

Aortic valve regurgitation differs from aortic valve stenosis in that patients with aortic valve regurgitation have unique anatomical features. Firstly, the patients with aortic valve regurgitation have little or no calcification of valve leaflets, and the valve leaflets are soft without any supporting effect. Secondly, the patients with aortic valve regurgitation have a larger valve ring and a larger ventricle, expanding with an ascending aorta. Therefore, several domestic valves specially designed for aortic valve stenosis are currently used for aortic valve regurgitation, which can only be anchored via several anchoring points of a valve ring, an outflow tract, a sinus canal junction area and the ascending aorta. However, the incidence of the problems of intraoperative valve displacement, valve-in-valve use and perivalvular regurgitation is still high.

A self-expanding valve suitable for aortic valve regurgitation and delivered through a femoral artery approach was disclosed in the present disclosure patent (Application NO. 2021115339418) filed by the applicant on Dec. 15, 2021, which can well solve the problems of valve displacement and perivalvular regurgitation. However, a first portion of the technical solution is corolla-shaped, with a long length. After being released, the first portion will extend into the ascending aorta, affecting the later coronary intervention, and such a valve cannot adjust a release position during release.

Therefore, a completely recoverable and self-expanding short-frame valve specifically for the patients with aortic valve regurgitation and released through the femoral artery approach is designed, realizing an one-time deployment in place with a stable releasing process, no post-operative migration and convenient later coronary intervention of the short frame, and realizing the complete recovery of the valve, which can solve the problem of minimally invasive treatment for many patients with aortic valve regurgitation and has broad market prospects.

### SUMMARY

In view of the defects in the prior art, the present disclosure provides a recoverable and self-expanding short-frame valve suitable for aortic valve regurgitation to solve the technical problems, which can realize the complete recovery of the valve without affecting the later coronary intervention.

In order to solve the above technical problems, the present disclosure provides a recoverable and self-expanding short-frame valve suitable for aortic valve regurgitation, including a tubular frame and valve leaflets sutured to an inner periphery of the tubular frame. The tubular frame has a plurality of hollow grids thereon to cause the tubular frame to contract to a delivery configuration or expand to a use configuration in a radial direction, the tubular frame includes a first portion and a second portion integrally formed and coaxially arranged, a waist portion is formed at the junction of the first portion and the second portion when the tubular frame is in the use configuration, and the second portion is divergently arranged in a direction away from the waist portion. The first portion in the direction away from the waist portion includes a divergent portion and an adduction portion, with an axial size ratio of the divergent portion to the adduction portion of 2:1; an axial size ratio of the second portion to the first portion is 1:3; and an end of the adduction portion away from the waist portion is formed with a plurality of endpoints, connection sections being arranged on at least five of the endpoints and configured to be detachably connected to steel wires of a valve delivery system.

According to the present disclosure, when in use, the tubular frame is contracted to its delivery configuration to form an elongated strip shape, and is delivered to an aortic valve via a femoral artery under the action of a delivery sheath; and after reaching a target position, the tubular frame expands to its use configuration. At this time, the waist portion is just aligned with an original valve ring, while the second portion anchors an outflow tract, and the second portion is divergently arranged in a direction away from the waist portion to stabilize the valve, thereby making it unable to be displaced in a direction away from the heart and making the whole valve release relatively stable. After the first portion is released, the divergent portion of the first portion is abutted against the valve ring to avoid later downward displacement of the valve. According to the present disclosure, the structure and size of the first portion are improved by designing the axial size ratio of the second portion to the first portion of 1:3, which shortens a length ratio of the first portion, so that after being released, the first portion will be completely within the sinus canal, that is, the adduction portion will not extend into an ascending aorta, without affecting the later coronary intervention. Moreover, connection sections are arranged on at least five of the endpoints and configured to be detachably connected to steel wires of the valve delivery system. If a release position of the valve is not matched or a model of the valve is not good, the complete recovery of the valve can be realized; and if the valve is well released, the steel wires of the delivery system can be separated from the connection sections to realize the complete release of the valve.

Preferably, the connection section is a stud integrally formed on the endpoints. Internal threads matching with the studs are correspondingly arranged on the steel wires of the valve delivery system, and the two are connected via the threads. When it is necessary to completely release the valve, the steel wires are rotated to separate the steel wires from the corresponding studs to realize the separation.

Preferably, the number of the endpoints is 5-10.

Preferably, a cross section where the junction of the adduction portion and the divergent portion is located is a first cross section, and a cross section where an end of the second portion away from the waist portion is located is a second cross section. A diameter of the first cross section is greater than that of the second cross section when the tubular frame is in the use configuration. With the design, the first portion can be effectively prevented from moving towards the heart to effectively fix the valve.

Preferably, the recoverable and self-expanding short-frame valve suitable for aortic regurgitation further includes an outer tectorial membrane covered on the peripheries of the second portion and a portion of the divergent portion near the waist portion. The design of the outer tectorial membrane can ensure that a portion of the valve in contact with tissues fits well, thereby reducing the perivalvular leakage.

Preferably, a divergence angle of the second portion in the direction away from the waist portion is 5°-10°.

Preferably, the tubular frame is made of a nickel-titanium alloy.

Preferably, a plurality of positioning mark points are uniformly distributed in a circumferential direction of the waist portion.

Preferably, the positioning mark point is made of gold.

Preferably, the outer tectorial membrane is made of PET.

### BRIEF DESCRIPTION OF THE DRAWINGS

In order to explain the specific implementations of the present disclosure or the technical solutions in the prior art more clearly, the attached drawings needed in the description of the specific implementations or the prior art will be briefly introduced below. In all attached drawings, similar elements or parts are generally identified by similar reference numerals. In the attached drawings, various elements or portions are not necessarily drawn to actual scales.

FIG. 1 is a schematic structural diagram of a recoverable and self-expanding short-frame valve suitable for aortic valve regurgitation according to an embodiment of the present disclosure; and

FIG. 2 is a schematic diagram of a connection structure of the recoverable and self-expanding short-frame valve suitable for aortic valve regurgitation and steel wires according to an embodiment of the present disclosure.

Reference numerals and denotations thereof:

1—first portion; 11—divergent portion; 12—adduction portion; 121—connection section; 2—second portion; 3—waist portion; 31—positioning mark point; 4—outer tectorial membrane; and 5—steel wire.

### DETAILED DESCRIPTION

Embodiments of the technical solutions of the present disclosure will be described in detail below with reference to the attached drawings. The following embodiments are only used to more clearly explain the technical solutions of the present disclosure, therefore, these embodiments are only used as examples, and cannot be used to limit the protection scope of the present disclosure.

It is noted that, unless otherwise indicated, technical or scientific terms used in the present application are to have the ordinary meanings as understood by those skilled in the art.

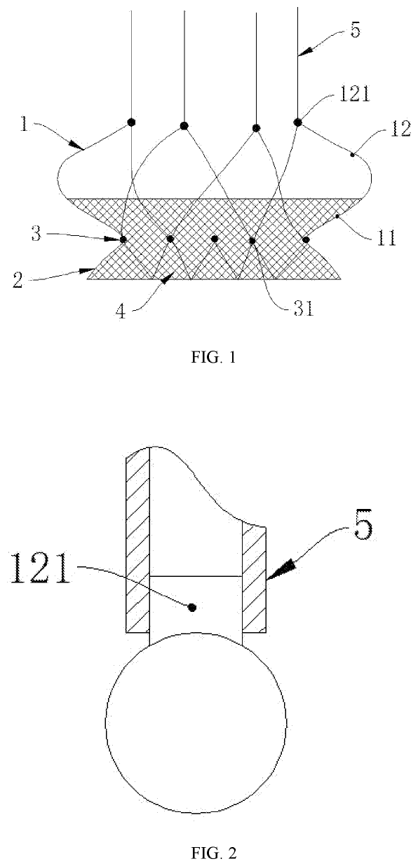

As shown in FIGS. 1 and 2, the embodiment provides a recoverable and self-expanding short-frame valve suitable for aortic valve regurgitation, including a tubular frame and three valve leaflets (not shown) sutured to an inner periphery of the tubular frame, the valve leaflets being made of porcine or bovine pericardium. The tubular frame has a plurality of hollow grids thereon to cause the tubular frame to contract to a delivery configuration or expand to a use configuration in a radial direction.

The above tubular frame includes a first portion 1 and a second portion 2 integrally formed and coaxially arranged, and a waist portion 3 is formed at the junction of the first portion 1 and the second portion 2 when the tubular frame is in the use configuration, and the above three valve leaflets are sutured to an inner periphery of the waist portion 3.

The above second portion 2 is divergently arranged in a direction away from the waist portion 3 with a divergence angle of 5°-10°. In the embodiment, the divergence angle is specifically 7°.

The above first portion 1 in the direction away from the waist portion 3 includes a divergent portion 11 and an adduction portion 12, with an axial size ratio of the divergent portion 11 to the adduction portion 12 of 2:1; and an axial size ratio of the second portion 2 to the first portion 1 of 1:3.

In the embodiment, the adduction portion 12 is designed to form an avoidance section that prevents an end portion of the adduction portion 12 from directly contacting with an aortic sinus to prevent the situation of puncture. The axial size ratio of the divergent portion 11 to the adduction portion 12 is designed to be 2: 1, which can improve the contact strength of the divergent portion 11 with a sinus wall and prevent the valve being displaced. Moreover, the axial size ratio of the second portion 2 to the first portion 1 is designed to be 1:3, to ensure, after being released, that the first portion 1 does not extend into the ascending aorta without affecting the later coronary intervention.

An outer tectorial membrane 4 is covered on the peripheries of the above second portion 2 and a portion of the divergent portion 11 near the waist portion 3. The outer tectorial membrane 4 is specifically made of a PET film, which can increase the frictional force, so that the tubular frame and a portion of the valve in contact with tissues fit well, thereby reducing the perivalvular leakage.

Further, an end of the above adduction portion 12 away from the waist portion 3 is formed with a plurality of endpoints. In practice, the number of the endpoints is 5-10. Connection sections 121 are arranged on at least five of the endpoints and configured to be detachably connected to steel wires 5 of a valve delivery system for the smooth delivery of the valve. Specifically, the above connection sections 121 are studs integrally formed on the endpoints, and internal threads matching with the stud threads are arranged on the steel wires 5 of the valve delivery system, and the two are connected via the threads. During release, if a release position of the valve is not matched or a model of the valve is not good, the complete recovery of the valve can be realized; and if the valve is well released, the steel wires 5 are rotated so that the steel wires 5 and the corresponding connection sections 121 are unthreaded, that is, the steel wires of the delivery system are separated from the connection sections 121 to realize the complete release of the valve.

Specifically, a cross section where the junction of the above adduction portion 12 and the divergent portion 11 is located is a first cross section, and a cross section where an end of the second portion 2 away from the waist portion 3 is located is a second cross section. A diameter of the first cross section is greater than that of the second cross section when the tubular frame is in the use configuration. With the design, the first portion 1 can be effectively prevented from moving towards the heart to effectively fix the valve. For example, in practice, if a diameter of the waist portion 3 is 35 mm, a diameter of the second cross section can be 40 mm and the diameter of the first cross section can be 45 mm, at this time, a length of the second portion 2 in an axial direction is 10 mm, and correspondingly a length of the first portion 1 in the axial direction is 25-30 mm. In the embodiment, the length of the first portion 1 in the axial direction is specifically 30 mm.

The tubular frame of the present embodiment is made of a nickel-titanium alloy, has the property of temperature memory, becomes soft in an ice-saline solution, and can be shaped and contracted from a use configuration to a delivery configuration. The tubular frame can self-expand at normal temperature (15-40° C., including blood at 370° C.) and revert to the designed shape (i.e. the shape of the use configuration), and has a high hardness. When in use, the tubular frame is contracted to its delivery configuration to form an elongated strip shape, the steel wires 5 of the delivery system are threadedly connected to the connection sections 121 of the valve and delivered to an aortic valve via a femoral artery under the action of a delivery sheath. After reaching a target position, the tubular frame is expanded to its use configuration. At this time, the waist portion 3 is just aligned with an original valve ring. Since the released waist portion 3 is at a same position as the original valve ring, the valve leaflets implanted into the valve is at a same position as original valve leaflets. The released second portion 2 anchors an outflow tract, and the second portion 2 is divergently arranged in a direction away from the waist portion 3 to stabilize the valve, thereby making it unable to be displaced in a direction away from the heart and making the whole valve release relatively stable. After the first portion 1 is released, the divergent portion 11 is abutted against the valve ring and a wall of the aortic sinus to avoid the later downward displacement of the valve to prevent puncture. After being completely released in place, the steel wires 5 are rotated so that the steel wires 5 are separated from the connection sections 121 to realize the complete release of the valve.

In order to smoothly align the waist portion with the original valve ring, a plurality of positioning mark points 31 are uniformly distributed in a circumferential direction of the waist portion 3 of the present embodiment, and the plurality of positioning mark points 31 are made of gold. During surgery, mark points can be designed at the original ring through contrast agents, and then upon the release of the valve, the positioning mark points 31 can be aligned with the designed mark points so that the waist portion 3 is just aligned with the original ring.

In the embodiment, the tubular frame is engraved by laser engraving technique, its hollow grids include first grids formed on the first portion 1 and second grids formed on the second portion 2, the first grids on the first portion 1 having a lower density than the second grids on the second portion 2. The grids of the first portion are relatively sparse, so that the whole valve ring stent can be loaded into a smaller delivery sheath in pressure, and the first portion 1 has such a relatively large radial cross section that the valve ring stent can be abutted against the wall of the aortic sinus, or a radial cross section size of the first portion 1 is close to that of the aortic sinus, which is beneficial to radial fixation without bias and enhance stability. The grids of the second portion 2 are relatively dense, and therefore, the valve ring stent has higher hardness and can provide better support.

Further, the above hollow grids have a rhombic shape, and a long axis direction of the rhombic hollow grids coincides with an axis direction of the tubular frame to form the elongated strip shape when the tubular frame is contracted to the delivery configuration.

In the description of the present disclosure, numerous specific details are set forth. However, it is understood that the embodiments of the present disclosure can be practiced without these specific details. In some instances, well-known methods, systems and techniques have not been shown in detail in order not to obscure the understanding of the specification.

In the description of the specification, referring to terms of “one embodiment”, “some embodiments”, “an example”, “a specific example”, or “some examples”, these descriptions mean that particular features, systems, materials, or characteristics described in connection with the embodiment or example are included in at least one embodiment or example of the present disclosure. In the specification, schematic representations of the above terms do not necessarily refer to the same embodiment or example. Furthermore, the particular features, systems, materials, or characteristics described can be combined in any suitable manner in any one or more embodiments or examples. Moreover, different embodiments or examples and features of different embodiments or examples described in the specification can be combined and composed by those skilled in the art without contradicting each other.

Finally, it is to be noted that: the above embodiments are merely illustrative of the technical solutions of the present disclosure, and are not limiting thereto; although the present disclosure has been described in detail with reference to the foregoing embodiments, those skilled in the art will appreciate that: the technical solutions recorded in the foregoing embodiments can still be modified or some or all of the technical features can be replaced by equivalents; such modifications or substitutions do not make the essence of the corresponding technical solutions deviate from the scope of the technical solutions of the embodiments of the present disclosure and are intended to be included within the scope of the claims and the specification of the disclosure.

1. A recoverable and self-expanding short-frame valve suitable for aortic valve regurgitation, comprising a tubular frame and valve leaflets sutured to an inner periphery of the tubular frame, the tubular frame having a plurality of hollow grids thereon to cause the tubular frame to contract to a delivery configuration or expand to a use configuration in a radial direction, the tubular frame comprising a first portion and a second portion integrally formed and coaxially arranged, a waist portion being formed at the junction of the first portion and the second portion when the tubular frame is in the use configuration, and the second portion being divergently arranged in a direction away from the waist portion, wherein:
the first portion in the direction away from the waist portion comprises a divergent portion and an adduction portion, with an axial size ratio of the divergent portion to the adduction portion of 2:1;
an axial size ratio of the second portion to the first portion is 1:3; and
an end of the adduction portion away from the waist portion is formed with a plurality of endpoints, connection sections being arranged on at least five of the endpoints and configured to be detachably connected to steel wires of a valve delivery system.

2. The recoverable and self-expanding short-frame valve suitable for aortic valve regurgitation according to claim 1, wherein:
the connection section is a stud integrally formed on the endpoints.

3. The recoverable and self-expanding short-frame valve suitable for aortic valve regurgitation according to claim 1, wherein:
the number of the endpoints is 5-10.

4. The recoverable and self-expanding short-frame valve suitable for aortic valve regurgitation according to claim 1, wherein:
a cross section where the junction of the adduction portion and the divergent portion is located is a first cross section, and a cross section where an end of the second portion away from the waist portion is located is a second cross section; and
a diameter of the first cross section is greater than that of the second cross section when the tubular frame is in the use configuration.

5. The recoverable and self-expanding short-frame valve suitable for aortic valve regurgitation according to claim 1, further comprising
an outer tectorial membrane covered on the peripheries of the second portion and a portion of the divergent portion near the waist portion.

6. The recoverable and self-expanding short-frame valve suitable for aortic valve regurgitation according to claim 1, wherein:
a divergence angle of the second portion in the direction away from the waist portion is 5°-10°.

7. The recoverable and self-expanding short-frame valve suitable for aortic valve regurgitation according to claim 1, wherein:
the tubular frame is made of a nickel-titanium alloy.

8. The recoverable and self-expanding short-frame valve suitable for aortic valve regurgitation according to claim 1, wherein:
a plurality of positioning mark points are uniformly distributed in a circumferential direction of the waist portion.

9. The recoverable and self-expanding short-frame valve suitable for aortic valve regurgitation according to claim 8, wherein:
the positioning mark point is made of gold.

10. The recoverable and self-expanding short-frame valve suitable for aortic valve regurgitation according to claim 5, wherein:
the outer tectorial membrane is made of PET.
